(19) United States
(12) Patent Application Publication
(10) Pub. No.: US 2006/0271071 A1
(43) Pub. Date: Nov. 30, 2006

(54) Epilation head for an epilation device

(76) Inventors: Ralf Dorber, Oberusel (DE); Pedro Sanchez-Martinez, Kronberg (DE)

Correspondence Address: FISH & RICHARDSON PC, P.O. BOX 1022, MINNEAPOLIS, MN, 55440-1022, US

(21) Appl. No.: 10/554,432

(22) PCT Filed: Apr. 14, 2004
(86) PCT No.: PCT/EP04/03900

§ 371 Date: Jun. 15, 2006

(30) Foreign Application Priority Data
Apr. 25, 2003 (DE) 103 18 749.9

(51) Int. Cl. A61B 17/50

(52) U.S. Cl. 606/133

(57) ABSTRACT

An epilation head for an epilator device, the epilation head comprising: a rotary body mounted for rotation about a longitudinal axis thereof, having first and second ends and an outer circumference extending from the first end to the second end; a plurality of pairs of clamping elements arranged about the outer circumference of the body and defining an active plucking length along the axis, wherein the clamping elements are actuated to cycle between open and gripping positions as the rotary body rotates, each pair of clamping elements defining an opening width in its open position; and wherein a sum of the opening widths of all the pairs of clamping elements is at least as great as the active plucking length.

### TECHNICAL FIELD

This invention relates to an epilation head for an epilator device.

### BACKGROUND

Epilator devices equipped with epilation heads are known in the art and currently rank among the top products in the field of hair removal. However, many of the epilation devices currently on the market require the user to move across some parts of the skin repeatedly until the last hair is removed from this particular skin surface. Some of the epilation heads employed in theses devices have been constructed as rotary bodies mounted for rotation about a longitudinal axis thereof, having a plurality of pairs of clamping elements distributed along the longitudinal axis and defining an active plucking length, wherein the clamping elements are actuated to cycle between open and gripping positions as the rotary body rotates in order to grip and pluck hairs between the clamping elements, each pair of clamping elements defining an opening width in its open position.

As an example, an epilation head of this type is described in WO 98/05234 in which a plurality of pairs of clamping elements are arranged on the circumference of a rotary cylinder in angularly offset relation to each other. In this arrangement, a total of 20 pairs of clamping elements are provided, each pair forming tweezers. The offset arrangement of the pairs of clamping elements about the axis of rotation of the rotary cylinder is such that after every one tenth of the full circle, that is, after every 36 degrees, two tweezers are simultaneously actuated in the closing direction.

During rotation of the rotary cylinder the individual pairs of clamping elements move along parallel circular paths which are spaced from each other laterally, that is, in the direction of the longitudinal axis of the rotary cylinder. With the rotary cylinder rotating, the tweezers reach the zone referred to as the plucking zone in open condition in which their two clamping elements are in spaced relation to one another by the opening width. As soon as a defined angular position is reached, the tweezers are moved towards one another by suitable actuating elements and brought into clamping engagement with one another. This closing instant or closing angle is identical for all the tweezers.

Owing to the lateral distance between the paths of motion of the individual tweezers, the overlap of their active plucking length, that is, the area bounded by the two outboard tweezers, is not complete. To ensure that hairs lying between the planes of motion of the tweezers are also fed to these tweezers, the envelope surface of the rotary cylinder is provided with inclined threading grooves which deflect the hairs in the direction of the tweezers such as to enable them to be captured by the clamping elements. However, it is still necessary, under the circumstances, to move across some parts of the skin repeatedly in order to achieve the desired epilation effect.

In addition, in epilation heads of this type, the plucking of hairs fed to the tweezers through the threading grooves occasionally causes problems in regard to the sense of pain, because during plucking a tug is exerted on these hairs at an angle to the skin surface which is not optimal with a view to avoiding or at least reducing pain. For example, EP 0 513 900 A2 discloses an epilation head of the type described above; however due to the thickness of the clamping elements to be taken into account the sum of the opening widths of all the pairs of clamping elements is smaller than the plucking length of the epilation head.

It is therefore desirable to further improve upon an epilation head of the type initially referred to in terms of both efficiency and reduced pain level.

### SUMMARY

In one aspect, the invention features an epilation head for an epilator device. The epilation head includes: a rotary body mounted for rotation about a longitudinal axis thereof, having first and second ends and an outer circumference extending from the first end to the second end; a plurality of pairs of clamping elements arranged about the outer circumference of the body and defining an active plucking length along the axis, wherein the clamping elements are actuated to cycle between open and gripping positions as the rotary body rotates, each pair of clamping elements defining an opening width in its open position; and wherein a sum of the opening widths of all the pairs of the clamping elements is at least as great as the active plucking length.

In some embodiments, the epilation head includes several rotary bodies each having respective clamping elements, wherein the sum of the opening widths of all the pairs of clamping elements on all the rotary bodies is at least as great as the sum of the active plucking lengths of all the rotary bodies of the epilation head.

In some cases, the clamping elements are distributed along the active plucking length at a uniform axial spacing. Furthermore, the clamping elements can be uniformly distributed over the outer circumference of the rotary body. In some variations of this aspect, the lateral offset between pairs of clamping elements succeeding each other in the direction of the longitudinal axis is smaller than or equal to the opening width. In addition, in some examples, the pairs of clamping elements succeeding each other in the circumferential direction of the rotary body can be positioned in overlapping relation to one another in the circumferential direction.

In some embodiments, the rotary body is formed of plural sections arranged axially one behind the other, and pairs of clamping elements, which are assigned to different sections, are brought into clamping engagement with one another in succession. In some cases, the clamping elements of each section are uniformly distributed in the axial direction. In addition, in some cases, the clamping elements of a section are uniformly distributed in the circumferential direction.

In yet another embodiment, the actuation of the clamping elements takes place such that at a defined instant of time only a single one of the plurality of pairs of clamping elements is brought into clamping engagement.

In some cases, the clamping elements are strip-shaped and are arranged perpendicular to the longitudinal axis in both longitudinal and circumferential directions. Such embodiments may include clamping elements having enlarged end portions extending in circumferential direction and bent up in a funnel-shaped configuration, wherein the distance between the end portions of each clamping element defines a gripping region.

In another aspect, the invention provides an epilation head for an epilator device. The epilation head includes: rotary body comprising a plurality of identical disk-shaped rotary components, each rotary component mounted adjacent to another about a common axis of rotation to form an essentially cylindrical structure; and a plurality of pairs of clamping elements arranged between the adjacent surfaces of each rotary component and distributed radially about a perimeter of the body at a substantially uniform axial spacing and defining an active plucking length along the axis of the body, wherein the clamping elements are actuated to cycle between open and gripping positions as the rotary body rotates about the axis of rotation, each pair of clamping elements defining an opening width in its open position; wherein a sum of the opening widths of all the pairs of the clamping elements is at least as great as the active plucking length.

In some cases, the disk-shaped rotary components are mounted in off-set relation to one another by a defined angle forming the rotary body with the pairs of clamping elements succeeding each other in a circumferential direction of the rotary body in overlapping relation to one another in the circumferential direction.

In some embodiments, the sum of the opening widths of all the pairs of clamping elements is greater than the active plucking length.

In some cases, the epilation head includes a lateral offset between pairs of clamping elements succeeding each other in the direction of the axis of rotation that is smaller than or equal to the opening width. The actuation of the clamping elements takes place such that at a defined instant of time only a single one of the plurality of pairs of clamping elements is brought into clamping engagement.

In another embodiment, the clamping elements are strip-shaped and are arranged perpendicular to the axis of rotation in both longitudinal and circumferential directions. In some variations of this embodiment, the clamping elements have enlarged end portions extending in circumferential direction and bent up in a funnel-shaped configuration, and wherein the distance between the end portions of each clamping element defines a gripping region.

In yet another aspect, the invention provides a method of epilating hair. The method includes: providing an epilator device, said epilator device having a housing, said housing containing a motor and an epilation head, which is mechanically driven by the motor, wherein the epilation head is exposed through an open end of the device housing, said epilation head comprising: a rotary body mounted for rotation about a longitudinal axis thereof, having first and second ends and an outer circumference extending from the first end to the second end; a plurality of pairs of clamping elements arranged about the outer circumference of the body and defining an active plucking length along the axis, wherein the clamping elements are actuated to cycle between open and gripping positions as the rotary body rotates, each pair of clamping elements defining an opening width in its open position; and wherein a sum of the opening widths of all of the pairs of the clamping elements is at least as great as the active plucking length; and passing the open end of said epilator device over an area of skin from which hair is to be epilated.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of an epilation head constructed in accordance with the present invention; and

FIG. 2 is a schematic developed view of the envelope surface of the rotary body, showing the location of the pairs of clamping elements carried by it.

### DETAILED DESCRIPTION

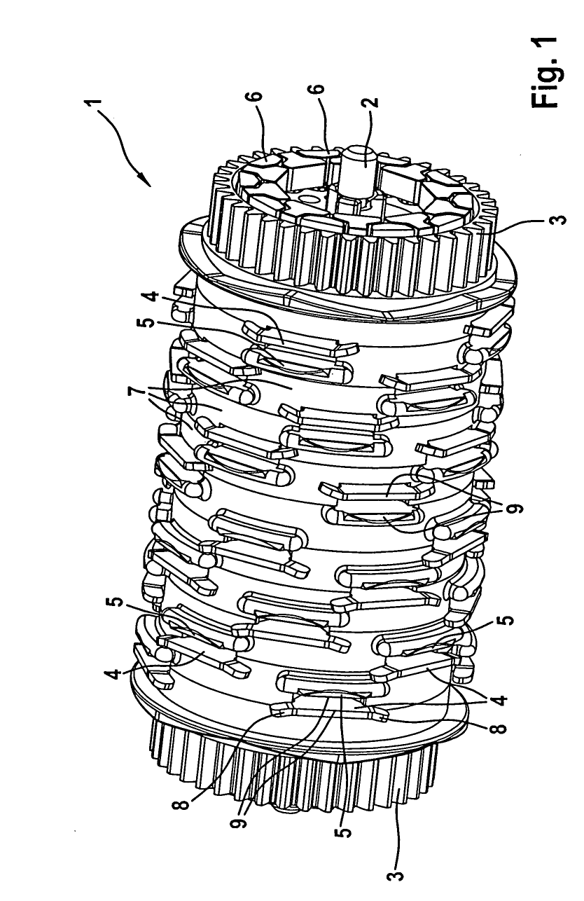

The epilation head 1 shown in FIG. 1 is associated with an epilator device having a housing containing a motor, and it has a rotary axle, which sits concentrically about a rotary axis 2, for rotatably carrying it in suitable bearings. The housing and the bearing arrangements receiving the epilation head are not shown in the drawings for greater clarity of the illustration. The epilation head 1 is provided with gears 3 for introduction of the torques. The epilation head 1 has a plurality of clamping elements 4, 5, with operatively associated clamping elements combining to form pairs of clamping elements. Each pair of clamping elements forms a tweezers composed of one movable clamping element 4 and one stationary clamping element 5. Actuation of the movable clamping elements 4 is in a manner known in the art from WO 98/05234, using actuating rods 6 mounted longitudinally displaceably in the interior of the epilation head parallel to its rotary axis 2. The actuating rods 6 lie on a circle extending concentrically with the rotary axis 2 and protrude a small amount from either end of the epilation head. They are controlled by cams arranged on the side of the epilation head 1, such that at a defined instant of time or at a defined angular position of the epilation head the actuating rods 6 execute a longitudinal displacement in the closing direction of their associated clamping elements 4. Being known in the art, this type of cam control is not shown in the Figures. With a view toward avoiding or minimizing pain during epilation in the best possible manner, it is an advantage for the actuation of the clamping elements to take place such that at a defined instant of time only a single one of the plurality of pairs of clamping elements is brought into clamping engagement. Experience has shown that the subjective sense of pain is significantly reduced when the individual plucking events occur in succession, compared to the simultaneous occurrence of plural plucking events. In this context, it is of primary importance, with a view to minimizing pain, for the elapsed time between individual plucking events to be as short as possible.

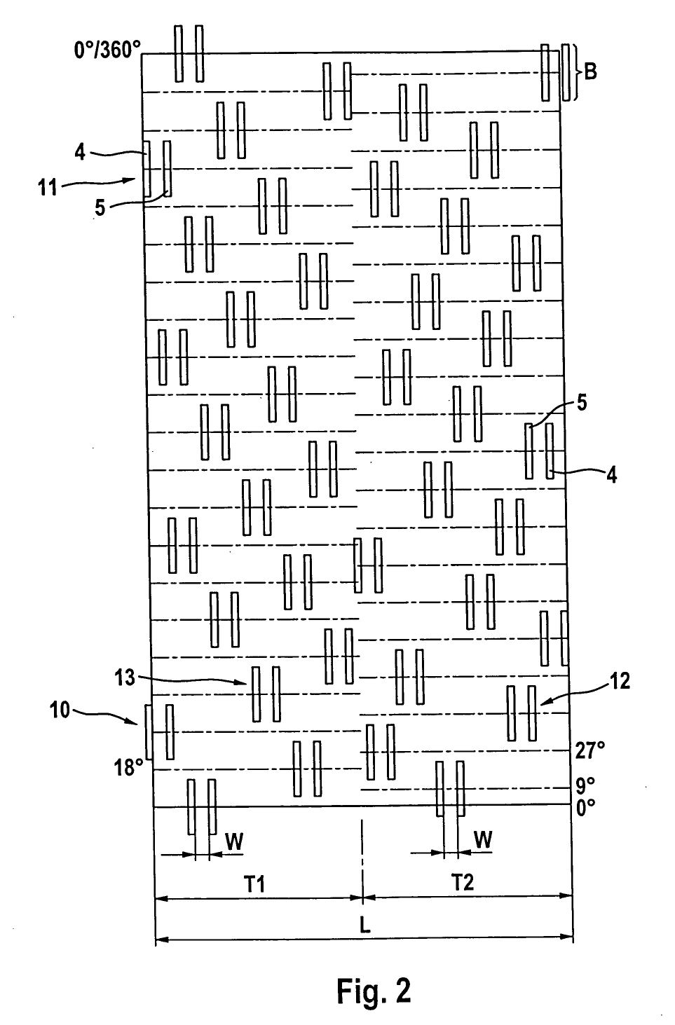

The clamping elements 4 and 5 are disposed in pairs between disk elements 7 arranged axially one behind the other. Regarding the adoption of a modular design principle using a plurality of identical disk-shaped elements, it is an advantage for the rotary body to be formed of plural sections arranged axially one behind the other, and for pairs of clamping elements 4, 5, which are assigned to different sections, to be brought into clamping engagement with one another in succession. In consequence, successive plucking events lie spatially apart by a minimum amount, whereby a reduction of the subjectively felt pain may be accomplished. Advantageously, the pairs of clamping elements 4, 5 are uniformly distributed both in the direction of the longitudinal (rotary) axis 2 and in the circumferential direction of the epilation head, including a defined lateral offset in the longitudinal direction and a defined angular offset in the circumferential direction. This is shown clearly and in detail in FIG. 2.

The strip-shaped clamping elements 4, 5 have enlarged end portions 8 extending in circumferential direction and bent up in a funnel-shaped configuration for improved gripping and threading in of the hairs and are designed to improve the feeding of the hairs to be plucked to the clamping location. Lying between these end portions 8 is the gripping region 9 with which the two clamping elements make relative contact when in the closed position. In open condition the gripping regions 9 of a pair of clamping elements 4, 5, which regions extend over a width B, are spaced from each other by the opening width W.

From the developed view of the envelope surface of the rotary body shown in FIG. 2, the distribution of the plurality of pairs of clamping elements 4, 5 becomes apparent. In axial direction of the rotary axis 2, the pairs of clamping elements are arranged with a defined lateral offset relative to each other. This lateral offset of pairs of clamping elements succeeding each other in axial direction, for example, of the tweezers 10 and 11, corresponds approximately to the thickness of a strip-shaped clamping element. This thickness is somewhat less than the opening width W. With the clamping elements positioned in this manner it is possible to achieve complete overlapping of the circular paths of all the pairs of clamping elements, so that, in all probability, all the hairs present on a skin section in need of epilating are extracted in one pass of the epilation head. This eliminates the need for the device to travel across one and the same location several times, thereby increasing user comfort and efficiency significantly.

Two pairs of clamping elements succeeding each other in circumferential direction, for example, the tweezers 12 and 13, are offset relative to one another about the rotary axis 2 by a defined angle. This angle amounts to 9 degrees in the embodiment, which is due to the fact that a total of 40 pairs of clamping elements 4, 5 are provided in uniformly distributed relation to one another around the circumference.

In respect of its axial dimension the epilation head 1 is composed of two sections T1 and T2 of equal size, whose dimension corresponds to half the active plucking length L. In each section the distribution of the tweezers both circumferentially and axially is uniform. The pairs of clamping elements 4, 5 of each section are offset relative to each other by an angle of 18 degrees. The amount of angular offset between the two sections T1 and T2 is 9 degrees. As a result of this distribution, tweezers actuated in succession as, for example, the tweezers 12 and 13, are assigned different sections T1 and T2, respectively.

In the transition region between the two sections T1 and T2 the lateral (axial) distance between the two inner lying pairs of clamping elements is even slightly reduced, resulting in a particularly high efficiency in the mid-region. This is an advantage when plucking individual hairs because the user intuitively directs the mid-region against these.

Considering that the lateral or axial offset of the tweezers is smaller or not greater than the opening width W, the total active plucking length L is covered by the circular paths of the pairs of clamping elements 4, 5. If the amount of axial offset is smaller than the opening width W, the circular paths will even be in overlapping relation. On account of the geometrical arrangement of the clamping elements 4, 5 shown in FIG. 2, the sum of the opening widths W of all the 40 pairs of clamping elements is greater than the active plucking length L; this makes it possible to achieve multiple overlapping of the epilation area with the pairs of clamping elements in comparatively simple manner and without particularly high expense.

The width B of the clamping elements is selected so that tweezers succeeding each other in circumferential direction—for example, the tweezers 12 and 13—are in overlapping relationship circumferentially. The sum of the tweezers widths of all the 40 tweezers is significantly greater than the circumference of the epilation head.

The at least 100% coverage of the active plucking length L by the pairs of clamping elements enables a highly efficient epilating operation to be accomplished. This efficiency may be increased still further when the epilator device is equipped with several such epilation heads in parallel arrangement. By virtue of the fact that each point along the active plucking length is covered by a pair of clamping elements, it is possible to capture the hairs in clamping relation optimally and as closely a possible to the hair root and to pluck them out using a straight plucking motion extending as perpendicularly to the skin surface as possible; this practically prevents the pain of epilation from occurring.

1. An epilation head for an epilator device, the epilation head comprising:
a rotary body mounted for rotation about a longitudinal axis thereof, having first and second ends and an outer circumference extending from the first end to the second end;
a plurality of pairs of clamping elements arranged about the outer circumference of the body and defining an active plucking length along the axis, wherein the clamping elements are actuated to cycle between open and gripping positions as the rotary body rotates, each pair of clamping elements defining an opening width in its open position; and
wherein a sum of the opening widths of all the pairs of the clamping elements is at least as great as the active plucking length.

2. The epilation head as claimed in claim 1, comprising several rotary bodies having respective clamping elements, wherein the sum of the opening widths of all the pairs of clamping elements on all the rotary bodies is at least as great as the sum of the active plucking lengths of all the rotary bodies of the epilation head.

3. The epilation head as claimed in claim 1 wherein the sum of the opening widths of all the pairs of clamping elements is greater than the active plucking length.

4. The epilation head according to claim 1, wherein the clamping elements are distributed along the active plucking length at a substantially uniform axial pacing.

5. The epilation head according to claim 1, wherein the clamping elements are uniformly distributed over the outer circumference of the rotary body.

6. The epilation head according to claim 1, wherein a lateral offset between pairs of clamping elements succeeding each other in the direction of the longitudinal axis is smaller than or equal to the opening width.

7. The epilation head according to claim 1, wherein the pairs of clamping elements succeeding each other in a circumferential direction of the rotary body are in overlapping relation to one another in the circumferential direction.

8. The epilation head according to claim 1, wherein the rotary body is formed of plural sections arranged axially one behind the other, and wherein pairs of clamping elements, which are assigned to different sections, are brought into clamping engagement with one another in succession.

9. The epilation head as according to claim 8, wherein the clamping elements of each section are uniformly distributed in axial direction.

10. The epilation head according to claim 8, wherein the clamping elements of a section are uniformly distributed in the circumferential direction.

11. The epilation head according to claim 1, wherein only a single pair of clamping elements is actuated into the gripping position at a defined instant of time.

12. The epilation head according to claim 1, wherein the clamping elements are strip-shaped and are arranged perpendicular to the longitudinal axis in both longitudinal and circumferential directions.

13. The epilation head according to claim 12, wherein the clamping elements have enlarged end portions extending in circumferential direction and bent up in a funnel-shaped configuration, and wherein the distance between the end portions of each clamping element defines a gripping region.

14. An epilation head for an epilator device, the epilation head comprising:
a rotary body comprising a plurality of identical disk-shaped rotary components, each rotary component mounted adjacent to another about a common axis of rotation to form an essentially cylindrical structure; and
a plurality of pairs of clamping elements arranged between the adjacent surfaces of each rotary component and distributed radially about a perimeter of the body at a substantially uniform axial spacing and defining an active plucking length along the axis of the body, wherein the clamping elements are actuated to cycle between open and gripping positions as the rotary body rotates about the axis of rotation, each pair of clamping elements defining an opening width in its open position;
wherein a sum of the opening widths of all the pairs of the clamping elements is at least as great as the active plucking length.

15. The epilation head according to claim 14, wherein the disk-shaped rotary components are mounted in off-set relation to one another by a defined angle forming the rotary body with the pairs of clamping elements succeeding each other in a circumferential direction of the rotary body in overlapping relation to one another in the circumferential direction.

16. The epilation head according to claim 14, wherein the sum of the opening widths of all the pairs of clamping elements is greater than the active plucking length.

17. The epilation head according to claim 14, wherein a lateral offset between pairs of clamping elements succeeding each other in the direction of the axis of rotation is smaller than or equal to the opening width.

18. The epilation head according to claim 14, wherein only a single pair of clamping elements is actuated into the gripping position at a defined instant of time.

19. The epilation device according to claim 14, wherein the clamping elements are strip-shaped and are arranged perpendicular to the axis of rotation in both longitudinal and circumferential directions.

20. The epilation head according to claim 14, wherein the clamping elements have enlarged end portions extending in circumferential direction and bent up in a funnel-shaped configuration, and wherein the distance between the end portions of each clamping element defines a gripping region.

21. A method of epilating hair, the method comprising:
providing an epilator device, said epilator device having a housing, said housing containing a motor and an epilation head, which is mechanically driven by the motor, wherein the epilation head is exposed through an open end of the device housing, said epilation head comprising:
a rotary body mounted for rotation about a longitudinal axis thereof, having first and second ends and an outer circumference extending from the first end to the second end;
a plurality of pairs of clamping elements arranged about the outer circumference of the body and defining an active plucking length along the axis, wherein the clamping elements are actuated to cycle between open and gripping positions as the rotary body rotates, each pair of clamping elements defining an opening width in its open position; and
wherein a sum of the opening widths of all of the pairs of the clamping elements is at least as great as the active plucking length; and
passing the open end of said epilator device over an area of skin from which hair is to be epilated.
